(19) United States
(12) Patent Application Publication
(10) Pub. No.: US 2022/0118041 A1
(43) Pub. Date: Apr. 21, 2022

(54) Blend of Plant-Extracted Oils

(71) Applicant: Johnson Consulting, LLC

(72) Inventor: Benjamin Johnson, Evergreen, CO (US)

(21) Appl. No.: 17/451,105

(22) Filed: Oct. 15, 2021

Related U.S. Application Data
(60) Provisional application No. 63/198,413, filed on Oct. 16, 2020.

(51) Int. Cl. A61K 36/72; A61K 31/201; A61K 31/353; A61K 36/185; A61K 31/202; A61K 31/20; A61K 31/575; A61K 31/232

(52) U.S. Cl. CPC A61K 36/72; A61K 31/201; A61K 31/353; A61K 31/232; A61K 31/202; A61K 31/20; A61K 31/575; A61K 36/185

(57) ABSTRACT

A plant-extracted oil oral supplement for skin repair. An example supplement includes a combination of sea buckthorn oil with at least one phytonutrient to reload adipocytes and thereby restore volume to fat pads in the human body. The supplement may also include palmitelaidic acid from sea buckthorn fruit oil and epigallocatechin from sea buckthorn seed oil and/or sea buckthorn pulp oil to improve conversion of calcium ATP-ase to magnesium ATP-ase and facilitate absorption of epigallocatechin (EPCG) into adipocytes corresponding to the fat pads.

### PRIORITY CLAIM

This application claims the priority filing benefit of U.S. Provisional Patent Application No. 63/198,413 filed Oct. 16, 2020 of Benjamin Johnson for “Blend of Plant-Extracted Oils,” hereby incorporated by reference for all that is disclosed as though fully set forth herein.

### BACKGROUND

A vast majority of health conditions and even diseases are the direct result of specific nutrient shortages that come about from chronic inflammation, toxicity, pathogen utilization, infection, and/or poor dietary habits.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1-8 are photographs showing before treatment (left) and after treatment (right).

### DETAILED DESCRIPTION

Sea Buckthorn extracts from the fruit, seed and pulp are each unique in their constitution. The breakdown of their individual antioxidants, nutrients, lipids, and omega fatty acids make it clear that the health benefits vary depending on the source of the sea buckhorn being ingested. Additionally, similar but still unique benefits can be had by other oils including macadamia nut oil.

When combined in a specific way, the sea buckthorn oil blends achieve remarkable benefits that have heretofore been undiscovered and do not happen with the individual oils themselves. Benefits may include, but are not limited to, repair of fat pad atrophy in the face and body, the treatment of high cholesterol, the treatment of diabetes, the clearance of allergies, improvements in high blood pressure, the improvement of skin conditions like acne, eczema, psoriasis, seborrheic dermatitis, perioral dermatitis, improvements in astigmatism, and more.

In addition, this unique blend of nutrients resolves digestive conditions such as, but not limited to, SIBO, H. pylori, chronic constipation, acid reflux, to name only a few examples.

Before continuing, it is noted that as used herein, the terms “includes” and “including” mean, but is not limited to, “includes” or “including” and “includes at least” or “including at least.” The term “based on” means “based on” and “based at least in part on.”

It is also noted that the examples described herein are provided for purposes of illustration, and are not intended to be limiting. Other devices and/or device configurations may be utilized to carry out the operations described herein.

The operations shown and described herein are provided to illustrate example implementations. It is noted that the operations are not limited to the ordering shown. Still other operations may also be implemented.

A vast majority of health conditions and even diseases are the direct result of specific nutrient shortages that come about from chronic inflammation, toxicity, pathogen utilization and/or poor dietary habits. By addressing these nutrient shortages at the same time, the body is able to heal damage that is otherwise not possible. This is referred to herein as the “bottleneck” theory.

The ingestion of sea buckthorn and macadamia nut oil has been purported to have health benefits. This is not surprising, considering they are all great sources for omega fatty acids. However, no one has reported the benefits of combining the various nutrient rich formulas in such a way to trigger repair of the serious conditions utilizing extracts of specific sources as disclosed herein.

The human body is capable of repairing damage in remarkable and complex ways when it has the tools to do so. However, if some critical components are not present while others are, then a bottleneck occurs and the level of repair response is limited by the limiting actives missing or in short supply. It requires the right combination of nutrients to be presented to the body daily to allow for a specifically calculated level of repair.

By way of example, the skin needs both Vitamin C and oxygen to function. High levels of either one do not make up for any shortages of the other component and the skin will fail if either is missing or in short supply. In the same way, sea buckthorn fruit oil has effective quantities of palmitelaidic acid that is modestly beneficial on its own but cannot trigger more advanced repair activity, including fat pad recovery, unless it is partnered with epigallocatechin from sea buckthorn seed oil and/or pulp oil. For example, plasma adipocytes and facial fat pad adipocytes rely on Magnesium ATPase to improve absorption. By including proper levels of palmitelaidic acid from sea buckthorn fruit oil, we improve conversion Calcium ATP-ase to Magnesium ATPase, which facilitates the absorption of epigallocatechin (EPCG) into the adipocytes related to fat pads. EPCG normally shrinks fat cell size as this oil blend has been shown to do, but it has adipocyte revival qualities in subcutaneous fat cells for structural fat pads.

Fat pad atrophy results from a loss in critical components within the fat cell that leads to function and volume losses. They can be referred to as “Haversian glands,” and those are noted to lose their content over time. To restore the size, strategies were created using relatively rare phytonutrients to reload the adipocytes and thereby restore volume.

Fat pad atrophy is well documented but poorly understood. Adults typically see an increase in their body fat over time and yet they see losses in the size of the fat cells associated with their fibrous fat pads. These fat pads have a more structural role in the body, they shape the face, they are present intramuscularly throughout the body including the buttocks, arms and legs. There is also a fat pad within the breast that loses volume with pregnancy and breastfeeding that is also poorly understood.

Fat pad atrophy may relate to their role in detoxification. Certain foods in particular can accelerate the atrophy including processed sugars, alcohol, refined salt and other chemical salts. Over time, with repeated efforts in detoxification as evidenced by the inflammatory cytokines and macrophages found in these adipocytes, the fat pads lose critical nutrients. Examples include, but are not limited to, epigallocatechin, gamma linolenic acid, lignoceric acid, cis-vaccenic acid, and beta sitosterol to name a few.

These losses result in the fat pad going dormant and/or shrinking in size from chronic inflammation. The accelerated detoxification in breastfeeding and/or the manufacturing of milk accelerates the breast fat pad atrophy.

It can take decades for many to see substantial fat pad losses, but it occurs more rapidly in alcoholics and sugar-laden diets. Gaining weight can mask some of the losses but the weight results in the enlargement of subcutaneous fat cells, not fat pad fat cells. Another source of fat pad losses is in low body fat individuals. In their case, the fats in the fat pads become a source of energy/fuel when the body does not have enough subcutaneous fat for that purpose.

All of these fat pads are distinct from the extensive subcutaneous fat layer. By replacing lost nutrients critical to fat pad function, the specific blend re-ignites detoxification activity over a (typically) 3 month cycle, whereby the subject ingests about 2 tablespoons a day. The restored fat pad also takes on volume during this time.

The ingestion of high amounts of oils can cause weight gain. Weight gain can hide facial fat pad losses and cause enlargement of the breasts. This is distinctly different from the effects we have studied with our unique oil blend whereby the trial participants lost weight and had very specific improvements to the face that correlate to anatomical locations of fat pads, not an overall rounding as is seen with weight gain.

An example plant-extracted oil oral supplement includes a combination of sea buckthorn oil with at least one phytonutrient to reload adipocytes and thereby restore volume to fat pads in the human body. The supplement may also include palmitelaidic acid from sea buckthorn fruit oil and epigallocatechin from sea buckthorn seed oil and/or sea buckthorn pulp oil to improve conversion of calcium ATP-ase to magnesium ATP-ase and facilitate absorption of epigallocatechin (EPCG) into adipocytes corresponding to the fat pads.

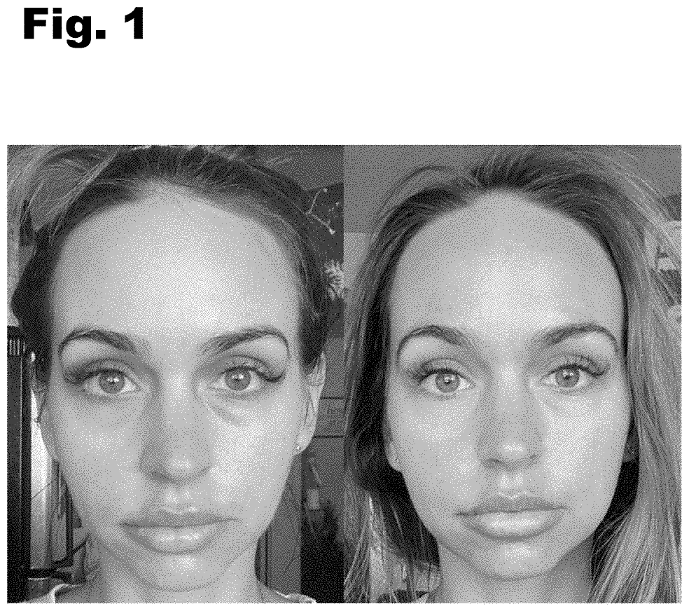

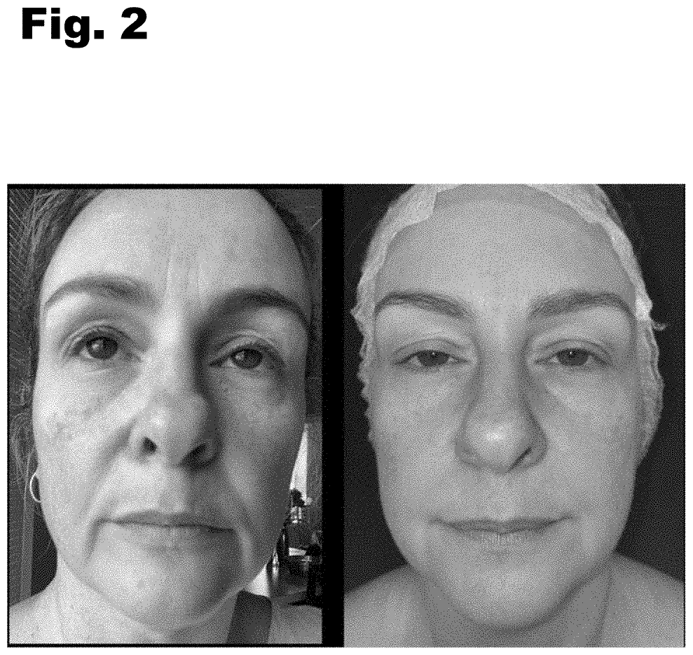

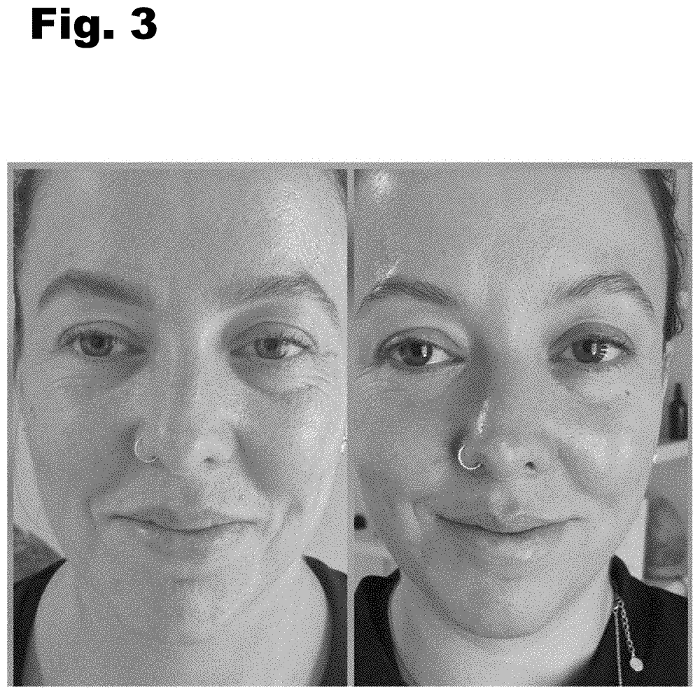

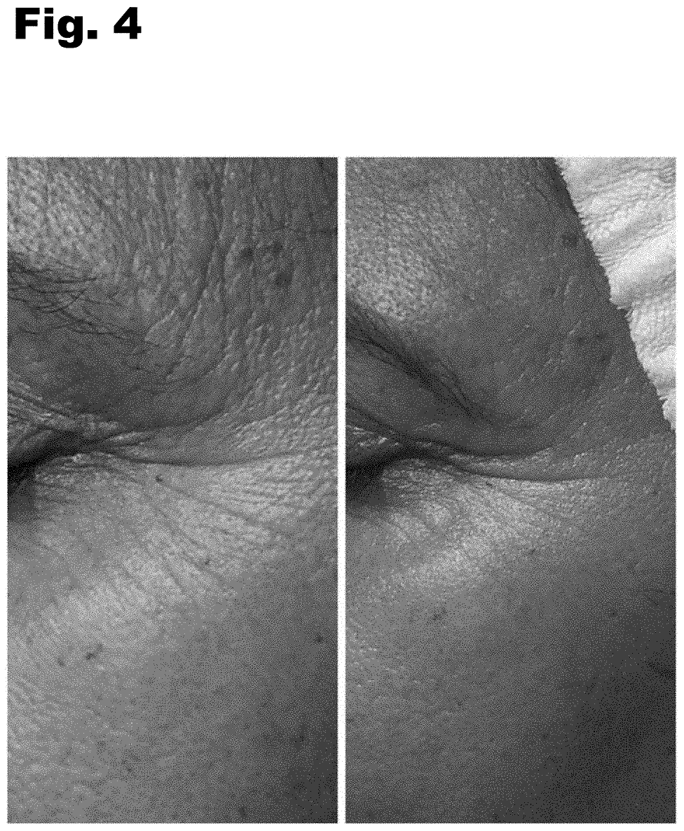

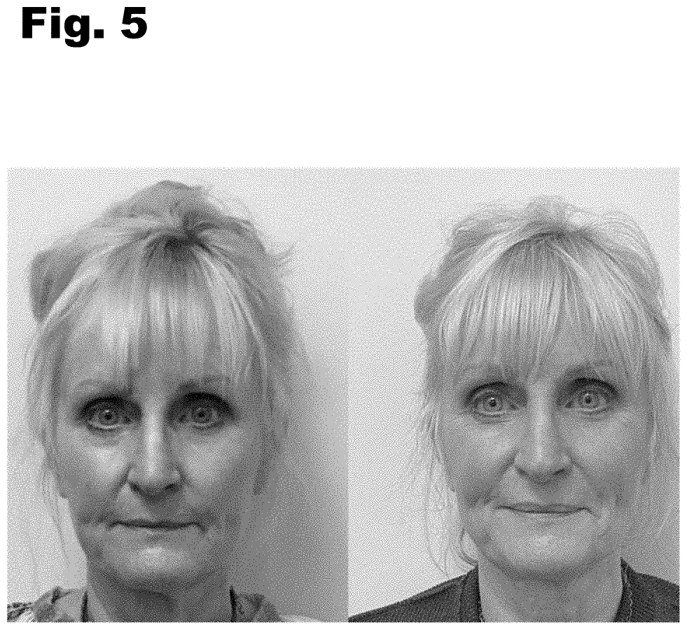

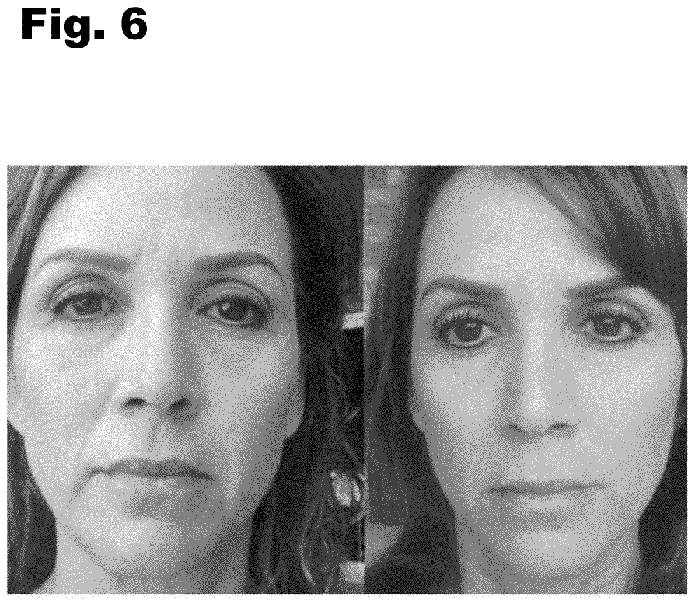

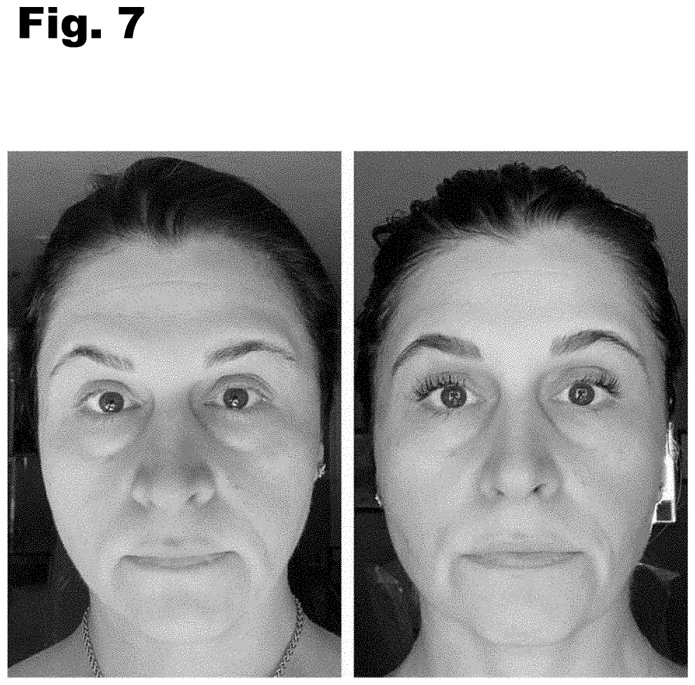

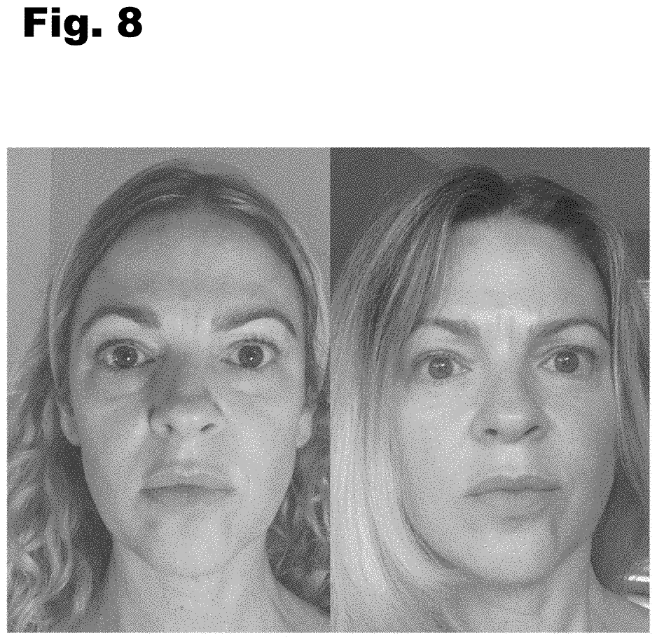

In an example, the supplement is administered over about a 3 month cycle at about 2 tablespoons per day. This re-ignites detoxification activity in the human body and the fat pads increase in volume during this cycle. FIGS. 1-8 are photographs showing before treatment (left) and after treatment (right) according to the supplement.

In an example, the supplement includes a macadamia nut oil. The macadamia nut oil may be provided in a range of 10-50 percent by weight relative to the sea buckthorn oil as determined by content of omega seven, behenic acid, and docosapentaenoic acid (DPA).

In an example, the supplement includes between about 10-50 percent by weight of sea buckthorn fruit oil as determined by content of palmitelaidic acid, eicosenoic acid, lignoceric acid, and beta sitosterol.

In an example, the supplement includes between about 10-50 percent by weight of sea buckthorn seed oil determined by measured amounts of gamma linolenic acid (GLA), alpha linolenic acid (ALA) and linoleic acid.

In an example, the supplement includes between about 5-40 percent by weight of sea buckthorn pulp oil determined by content of beta sitosterol, gallocatechin, epigallocatechin, cis-vaccenic acid and palmitoleic acid.

In an example, the supplement includes between about 2-10 percent by weight of gamma linolenic acid, between about 0.5-3 percent by weight of linoleic acid, and between about 0.5-3 percent by weight alpha linolenic acid in sea buckthorn seed oil.

In an example, the supplement includes between about 0.05-2 percent by weight palmitelaidic acid, between about 0.05-2 percent by weight eicosenoic acid, between about 0.01-1 percent by weight lignoceric acid, and between about 0.5-5 percent by weight beta sitosterol in sea buckthorn fruit oil.

In an example, the supplement includes between about 0.5-5 percent by weight beta sitosterol, between about 1-7 percent by weight gallocatechin, between about 0.5-5 percent by weight epigallocatechin, between about 3-12 percent by weight cis-vaccenic acid, and between about 0.1-4 percent by weight palmitoleic acid in sea buckthorn pulp oil.

In an example, the supplement includes between about 0.1-4 percent by weight beta docosapentoic acid, between about 0.05-2 percent by weight behenic acid, and between about 1-10 percent by weight 17-tetracosenoic acid in macadamia nut oil.

The plant-extracted oil oral supplement disclosed herein may also be prescribed to address a number of other ailments. By way of example, diabetes is still perplexing researchers. The proposed mechanism of action of the oil blend disclosed herein may be helpful in elucidating the real cause(s) of diabetes. The appropriate amounts of linoleic acid in combination with eicosanoic acid of this blend target a specific strain (S. thermophilus) in the microbiome to encourage better sugar utilization.

Cholesterol utilization is also the way the oil blend disclosed herein improves subjects with high blood levels of cholesterol. It is accelerated within the Firmicutes phylum because of the unique combination of appropriate levels and of palmitoleic acid and linoleic acid. This needs to be presented simultaneously to prevent the bottleneck effect described above.

SIBO and H pylori are phenotypic (epigenetic) changes of existing probiotics that result from harmful diets and poor digestive capacity. SB Blend provides the exact nutrients needed to reverse those epigenetic changes and restore healthy, functioning digestive bacteria. Specifically, the higher levels of arachidonic acid in sea buckthorn seed oil and macadamia nut oil presented synergistically with the specific blend of higher levels of luteolin in sea buckthorn fruit extract can convert the phenotype of the harmful forms of these bacteria.

High blood pressure can come from a few causes that are known (kidney damage), but many times it is poorly understood. SB Blend provides a specific nutrient combination that helps relax smooth muscle by affecting increasing Magnesium ATPase as previously discussed.

Allergies are impacted as well by SB Blend. This mechanism relates to repairing of the gut wall. Leaky gut is a common condition that results from chronic antibiotics, chemotherapy and other causes. SB blend provides the needed amount of aglycones in seed oil with the fruit oil derived higher levels of zeaxanthin to promote digestive lining repair and improve allergies by reducing leaky gut.

It is noted that the examples shown and described are provided for purposes of illustration and are not intended to be limiting. Still other examples are also contemplated.

1. A plant-extracted oil oral supplement for skin repair, the supplement comprising a combination of sea buckthorn oil with at least one phytonutrient to reload adipocytes and thereby restore volume to fat pads in the human body.

2. The supplement of claim 1, further comprising palmitelaidic acid from sea buckthorn fruit oil and epigallocatechin from sea buckthorn seed oil and/or sea buckthorn pulp oil to improve conversion of calcium ATP-ase to magnesium ATP-ase and facilitate absorption of epigallocatechin (EPCG) into adipocytes corresponding to the fat pads.

3. The supplement of claim 1, wherein the supplement administered over about a 3 month cycle at about 2 tablespoons per day re-ignites detoxification activity in the human body and the fat pads increase in volume during this cycle.

4. The supplement of claim 1, further comprising a macadamia nut oil in a range of 10-50 percent by weight relative to the sea buckthorn oil as determined by content of omega seven, behenic acid, and docosapentaenoic acid (DPA).

5. The supplement of claim 1, further comprising between about 10-50 percent by weight of sea buckthorn fruit oil as determined by content of palmitelaidic acid, eicosenoic acid, lignoceric acid, and beta sitosterol.

6. The supplement of claim 1, further comprising between about 10-50 percent by weight of sea buckthorn seed oil determined by measured amounts of gamma linolenic acid (GLA), alpha linolenic acid (ALA) and linoleic acid.

7. The supplement of claim 1, further comprising between about 5-40 percent by weight of sea buckthorn pulp oil determined by content of beta sitosterol, gallocatechin, epigallocatechin, cis-vaccenic acid and palmitoleic acid.

8. The supplement of claim 1, further comprising between about 2-10 percent by weight of gamma linolenic acid, between about 0.5-3 percent by weight of linoleic acid, and between about 0.5-3 percent by weight alpha linolenic acid in sea buckthorn seed oil.

9. The supplement of claim 1, further comprising between about 0.05-2 percent by weight palmitelaidic acid, between about 0.05-2 percent by weight eicosenoic acid, between about 0.01-1 percent by weight lignoceric acid, and between about 0.5-5 percent by weight beta sitosterol in sea buckthorn fruit oil.

10. The supplement of claim 1, further comprising between about 0.5-5 percent by weight beta sitosterol, between about 1-7 percent by weight gallocatechin, between about 0.5-5 percent by weight epigallocatechin, between about 3-12 percent by weight cis-vaccenic acid, and between about 0.1-4 percent by weight palmitoleic acid in sea buckthorn pulp oil.

11. The supplement of claim 1, further comprising between about 0.1-4 percent by weight beta docosapentoic acid, between about 0.05-2 percent by weight behenic acid, and between about 1-10 percent by weight 17-tetracosenoic acid in macadamia nut oil.

12. A plant-extracted oil oral supplement comprising:
a combination of sea buckthorn oil with at least one phytonutrient to reload adipocytes;
palmitelaidic acid from sea buckthorn fruit oil and epigallocatechin from sea buckthorn seed oil and/or sea buckthorn pulp oil to improve conversion of calcium ATP-ase to magnesium ATP-ase and facilitate absorption of epigallocatechin (EPCG) into adipocytes;
wherein the supplement is administered over about a 3 month cycle at about 2 tablespoons per day re-ignites detoxification activity in the human body and the fat pads increase in volume during this cycle.

13. The supplement of claim 12, further comprising between about 0.1-4 percent by weight beta docosapentoic acid, between about 0.05-2 percent by weight behenic acid, and between about 1-10 percent by weight 17-tetracosenoic acid in macadamia nut oil.

14. The supplement of claim 12, further comprising a macadamia nut oil in a range of 10-50 percent by weight relative to the sea buckthorn oil as determined by content of omega seven, behenic acid, and docosapentaenoic acid (DPA).

15. The supplement of claim 12, further comprising between about 10-50 percent by weight of sea buckthorn fruit oil as determined by content of palmitelaidic acid, eicosenoic acid, lignoceric acid, and beta sitosterol.

16. The supplement of claim 12, further comprising between about 10-50 percent by weight of sea buckthorn seed oil determined by measured amounts of gamma linolenic acid (GLA), alpha linolenic acid (ALA) and linoleic acid.

17. The supplement of claim 12, further comprising between about 5-40 percent by weight of sea buckthorn pulp oil determined by content of beta sitosterol, gallocatechin, epigallocatechin, cis-vaccenic acid and palmitoleic acid.

18. The supplement of claim 12, further comprising between about 2-10 percent by weight of gamma linolenic acid, between about 0.5-3 percent by weight of linoleic acid, and between about 0.5-3 percent by weight alpha linolenic acid in sea buckthorn seed oil.

19. The supplement of claim 12, further comprising between about 0.05-2 percent by weight palmitelaidic acid, between about 0.05-2 percent by weight eicosenoic acid, between about 0.01-1 percent by weight lignoceric acid, and between about 0.5-5 percent by weight beta sitosterol in sea buckthorn fruit oil.

20. The supplement of claim 12, further comprising between about 0.5-5 percent by weight beta sitosterol, between about 1-7 percent by weight gallocatechin, between about 0.5-5 percent by weight epigallocatechin, between about 3-12 percent by weight cis-vaccenic acid, and between about 0.1-4 percent by weight palmitoleic acid in sea buckthorn pulp oil.
